(19) United States
(12) Patent Application Publication
(10) Pub. No.: US 2017/0030913 A1
(43) Pub. Date: Feb. 2, 2017

(54) COMPOSITION OR KIT FOR DIAGNOSING COLORECTAL CANCER INCLUIDNG CXCL7-MEASURING AGENT AND METHOD OF DIAGNOSING COLORECTAL CANCER USING THE SAME

(71) Applicants: KOREA INSTITUTE OF SCIENCE AND TECHNOLOGY, Seoul (KR); SAMSUNG LIFE PUBLIC WELFARE FOUNDATION, Seoul (KR)

(72) Inventors: Ji Eun LEE, Seoul (KR); Dong Huey CHEON, Seoul (KR); Cheolju LEE, Seoul (KR); Eun Gyeong YANG, Seoul (KR); Jihye SHIN, Seoul (KR); Hee Cheol KIM, Seoul (KR); Ji Hye HAN, Seoul (KR)

(21) Appl. No.: 15/220,521

(22) Filed: Jul. 27, 2016

(30) Foreign Application Priority Data
Jul. 30, 2015 (KR) 10-2015-0108153

(51) Int. Cl. G01N 33/574

(52) U.S. Cl. CPC G01N 33/57419; G01N 2333/521; G01N 2560/00

(57) ABSTRACT

Provided are a composition or a kit for diagnosing colorectal cancer including a CXCL7-measuring agent, and a method of diagnosing colorectal cancer using the same. According to the composition or the kit, and the method, colorectal cancer may be simply diagnosed with high diagnostic accuracy and specificity for colorectal cancer.

### CROSS-REFERENCE TO RELATED APPLICATION

This application claims the benefit of Korean Patent Application No. 10-2015-0108153, filed on Jul. 30, 2015, in the Korean Intellectual Property Office, the disclosure of which is incorporated herein in its entirety by reference.

### BACKGROUND

1. Field

The present disclosure relates to a composition or a kit for diagnosing colorectal cancer, and a method of diagnosing colorectal cancer using the same.

2. Description of the Related Art

Colorectal cancer is a very common malignancy in the world including Korea. In Korea, colorectal cancer is the fourth most common malignancy in both women and men, and the death rate from colorectal cancer also ranked fourth with about 7 per 100,000 people. For the recent 10 years, the death rate has increased by about 80%. When colorectal cancer is detected at an early stage, its ultimate cure rate approaches 100%. In general, there are no distinctive subjective symptoms, and therefore, early detection is difficult.

For diagnosis of colorectal cancer, methods such as a colon contrast study, colonoscopy, radiodiagnosis, etc. are currently used. A colorectal cancer biomarker called Carcinoembryonic Antigen (CEA) is routinely used, but it is merely used as an index for progression of colorectal cancer and for evaluation of therapeutic effects. There have been no qualified biomarkers which can be used in blood tests for the early detection or prognosis of colorectal cancer.

To develop biomarkers in the blood, serum, or plasma, proteins present in the blood, serum, or plasma are separated, and the separated proteins are digested by using proteases, and then differentially expressed proteins are selected based on the identified peptides. According to these previous methods using proteases, however, information about peptide fragments is obtained, and therefore, the obtained information is only partial information about the proteins. It is difficult to identify cancer-specific protein isoforms by using only information of polypeptide fragments that are produced from digestion of proteins.

Accordingly, there is a need to develop biomarkers with high diagnostic accuracy and specificity, which may be used to diagnose colorectal cancer by a simple blood test.

### SUMMARY

An aspect provides a composition for diagnosing colorectal cancer.

Another aspect provides a kit for diagnosing colorectal cancer.

Still another aspect provides a method of diagnosing colorectal cancer.

### BRIEF DESCRIPTION OF THE DRAWINGS

These and/or other aspects will become apparent and more readily appreciated from the following description of the embodiments, taken in conjunction with the accompanying drawings in which:

FIG. 1A is an illustration of a gel-eluted liquid fraction entrapment electrophoresis (GELFrEE) device, and FIG. 1B is an image of a silver-stained gel of GELFrEE fractionation; and

FIG. 2A is a graph showing relative intensities of CXCL7 in a normal group and a colorectal cancer group according to immunoblotting results, and FIG. 2B is a receiver operating characteristic (ROC) curve of CXCL7 in the normal group and the colorectal cancer group.

### DETAILED DESCRIPTION

An aspect provides a composition for diagnosing colorectal cancer, the composition including an agent measuring an expression level of a chemokine (C-X-C motif) ligand 7 (CXCL7) protein or a fragment thereof.

The CXCL7 protein is a low-molecular weight cytokine belonging to the CXC chemokine family. The CXCL7 protein may be beta-thromboglobulin (p-thromboglobulin) or pro-platelet basic protein (PPBP). The CXCL7 protein may be also named PPBP, B-TG1, beta-TG, CTAP-III, CTAP3, CTAPIII, LA-PF4, LDGF, MDGF, NAP-2, PBP, SCYB7, TC1, TC2, TGB, TGB1, THBGB, or THBGB1. The CXCL7 protein may be a protein encoded by a nucleic acid including a nucleotide sequence of GenBank Accession No. NM_002704 in humans. The CXCL7 protein may be a protein including an amino acid sequence of GenBank Accession No. NP_002695 or an amino acid sequence of UniProt No. P02775 in humans. The CXCL7 protein may be a protein encoded by a nucleic acid including a nucleotide sequence of GenBank Accession No. NM_023785, or a protein including an amino acid sequence of GenBank Accession No. NP_076274 in mice. The CXCL7 protein may be a protein composed of 128 amino acids. Amino acid sequences at amino acid positions 1 to 34 from the N-terminus of the CXCL7 protein may be a signal peptide. A protein of amino acid sequences at amino acid positions 35 to 128, or 44 to 128 from the N-terminus of the CXCL7 protein may be the CXCL7 protein.

The CXCL7 protein may be a protein including an amino acid sequence of SEQ ID NO: 1. The CXCL7 protein or a fragment thereof may be a CXCL7 isoform protein. The term “isoform” refers to a different form of the same protein. The isoform may be different forms of protein coded from the same gene, or proteins with functional similarities, even when they are products of different genes. The CXCL7 isoform protein may be a protein including an amino acid sequence of SEQ ID NO: 2. The CXCL7 protein having the amino acid sequence of SEQ ID NO: 2 may be also called connective tissue-activating peptide III.

The CXCL7 protein or a fragment thereof may be an immunogenic fragment. The immunogenic fragment may be a fragment of the CXCL7 protein having one or more epitopes which may be recognized by antibodies against the CXCL7 protein.

The expression level may be a protein expression level. The expression level may be determined by measuring the amount or concentration of the protein in a sample. The expression level may be expressed as an absolute (e.g., μg/ml) or relative (e.g., relative intensity of signals) difference of the protein.

The amount of the CXCL7 protein or fragment thereof may be measured by electrophoresis, immunoblotting, enzyme-linked immunosorbent assay (ELISA), protein chips, immunoprecipitation, mass spectrometry, or a combination thereof.

The agent measuring the expression level refers to a substance that may be used for detection or quantification of the protein or the fragment thereof. The agent may be an antibody specifically binding to the CXCL7 protein or the fragment thereof or an antigen-binding fragment of the antibody. The term “antibody” may be used interchangeably with the term “immunoglobulin”. The antibody may be a polyclonal antibody or a monoclonal antibody. The antibody may be a full-length antibody. The antigen-binding fragment refers to a polypeptide including an antigen-binding site. The antigen-binding fragment may be a single-domain antibody, Fab, Fab′, or scFv. The antibody or antigen-binding fragment may be a commercially available antibody against the CXCL7 protein or antigen-binding fragment thereof.

The composition may further include a sample needed for the diagnosis of colorectal cancer.

Another aspect provides a kit for diagnosing colorectal cancer, the kit including the agent measuring the expression level of the CXCL7 protein or the fragment thereof.

The CXCL7 protein, the fragment of CXCL7 protein, the expression level, and the measurement of the expression level are the same as described above.

The kit may further include a sample needed for the diagnosis of colorectal cancer. The kit may include an antibody, a substrate for immunological detection of the antibody, an appropriate buffer solution, a coloring enzyme or fluorescent-labeled secondary antibody, or a coloring substrate.

Still another aspect provides a method of diagnosing colorectal cancer, the method including measuring an expression level of the CXCL7 protein or the fragment thereof in a biological sample separated from a subject suspected of having colorectal cancer; and comparing the measured expression level with a CXCL7 protein expression level of a normal control group.

The CXCL7 protein, the fragment of CXCL7 protein, the expression level, and the measurement of the expression level are the same as described above.

The method includes measuring the expression level of the CXCL7 protein or the fragment thereof in the biological sample separated from the subject suspected of having colorectal cancer.

The subject may be a mammal including a human.

The biological sample refers to a sample obtained from an organism. The biological sample may be blood, plasma, platelet, serum, ascitic fluid, bone marrow fluid, lymphatic fluid, saliva, lachrymal fluid, mucosal fluid, amniotic fluid, or a combination thereof.

The biological sample may include an intact protein. The intact protein may be a protein separated from the biological sample without additional modification of the protein. The intact protein may be a protein separated from the biological sample without, for example, protein degradation by protease.

The measuring an expression level of the CXCL7 protein or the fragment thereof in a biological sample separated from a subject suspected of having colorectal cancer may include incubating the biological sample and an antibody specifically binding to the CXCL7 protein or the fragment thereof. The incubating may be performed in vitro. The measuring method may be, for example, immunoblotting, ELISA, protein chips, immunoprecipitation, or a combination thereof.

The measuring may include analyzing the biological sample using mass spectrometry. The mass spectrometric analysis of the biological sample may be analyzing the intact protein using mass spectrometry.

The method includes comparing the measured expression level with the protein expression level of the normal control group.

The normal control group may be a biological sample separated from a subject who is not a patient with colorectal cancer, a subject having benign tumor, or a subject not being at the risk of having colorectal cancer.

When the expression level of the CXCL7 protein or the fragment thereof measured in the biological sample separated from the subject suspected of having colorectal cancer is higher than the expression level of the CXCL7 protein or the fragment thereof measured in the sample separated from the normal control group, the subject may be determined as a patient with colorectal cancer or at a high risk of having colorectal cancer.

The method includes a method of providing information for the diagnosis of colorectal cancer.

According to the composition or the kit for diagnosing colorectal cancer, and the method of diagnosing colorectal cancer using the same of an aspect, colorectal cancer may be simply diagnosed with high diagnostic accuracy and specificity for colorectal cancer.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

Hereinafter, the present invention will be described in more detail with reference to Examples. However, these Examples are for illustrative purposes only, and the scope of the present invention is not intended to be limited by these Examples.

### Example 1

### Identification of Differentially Expressed Proteins in Plasma Proteins of Colorectal Cancer Patients

1. Preparation of Plasma of Colorectal Cancer Patients and Healthy Controls

Plasma samples of 3 normal persons and plasma samples of 3 colorectal cancer patients were collected from Gene Banks of Seoul National University Bundang Hospital and Severance Hospital, respectively. To prevent degradation of proteins in the samples, a protease inhibitor, complete mini EDTA-free (Roche) and a phosphatase inhibitor, PhosSTOP (Roche) were added to the respective collected samples, immediately after collection of the samples or when the samples were first thawed. For mass spectrometry, equal amounts of plasma proteins from each sample were pooled, and then stored at −80° C. before use in experiments.

2. Separation of Plasma Proteins

2.1. Separation of Plasma Proteins Based on Molecular Weight

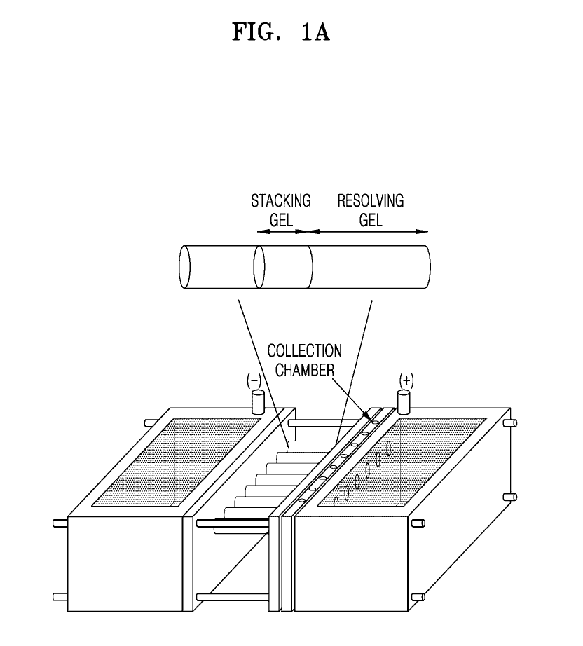

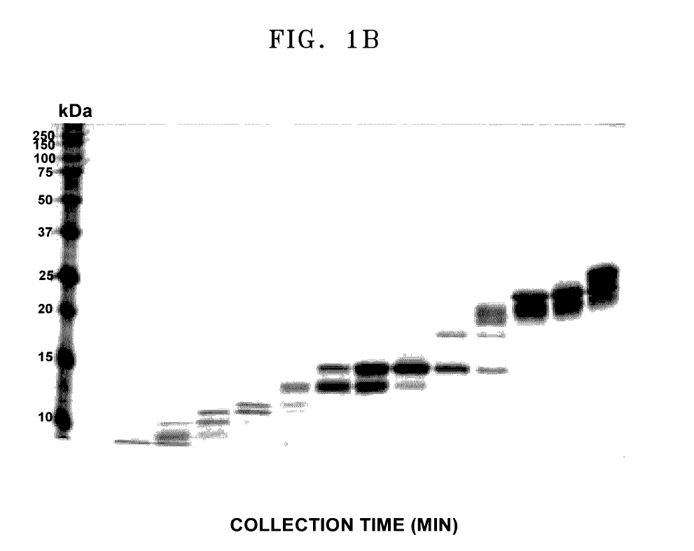

There is a difficulty in mass spectrometric analysis of the plasma samples, due to high complexity of proteins. To reduce protein complexity, proteins in the plasma samples were separated based on their molecular weights using a gel-eluted liquid fraction entrapment electrophoresis (GELFrEE) device (manufactured in-house based on “A. A., Multiplexed size separation of intact proteins in solution phase for mass spectrometry. Anal. Chem. 2009, 81, 6201-6209”) (FIG. 1A).

In detail, 850 μg of the plasma pooled in 1. was loaded on a glass column in a loading chamber of the GELFrEE device, followed by electrophoresis at a voltage of 240 V. The column was an SDS-polyacrylamide tube gel consisting of 4% of a stacking gel (1.5 cm) and 17.5% of a resolving gel (3 cm). Protein fractions were collected at each time point of 3 minutes, 6 minutes, 9 minutes, 11 minutes, 13 minutes, 15 minutes, 17 minutes, 20 minutes, 25 minutes, 30 minutes, 35 minutes, 40 minutes, 45 minutes, and 50 minutes after a blue dye was eluted from the column. The fractionation was then visualized by silver staining of a SDS-PAGE slab gel with 8 μL of each 150 μL GELFrEE fraction. An image of the silver-stained gel is shown in FIG. 1B.

As shown in FIG. 1B, it was confirmed that the plasma proteins were separated according to their molecular weights.

2.2. Concentration of Proteins

From the protein fractions, of which molecular weights were confirmed in 2.1., the GELFrEE fractions with similar molecular weight ranges collected from eight to 24 channel replicates of GELFrEE corresponding to one to three eight-channel multiplexed GELFrEE runs were typically combined and concentrated using an Amicon Ultracel-3 centrifugal filter (3 kDa cutoff) Prior to concentration of the fractionated samples, a centrifugal filter device was first rinsed two times with 450 μl of water 14,000×g for 10 min, and conditioned two times with 450 μl of 10 mM Tis-HCl (pH 7.5) at 14,000×g for 10 min. The concentrated samples having approximately 100 μl were recovered by inverting the filter device and centrifuged at 2000×g for 2 min.

2.3. Purification of Proteins

The concentrated protein samples were purified by methanol/chloroform/water precipitation.

In detail, 400 μl of methanol (HPLC grade, J.T baker) was added to the protein samples recovered in 2.2., followed by vortexing for 1 minute. 100 μl of chloroform (Sigma-Aldrich) was added to the mixture, followed by vortexing for 1 minute. Thereafter, 300 μl of distilled water was added to the mixture, followed by vortexing for 1 minute. The mixture was centrifuged at 10° C. and at a speed of 13,000 rpm for 20 minutes. A supernatant was carefully discarded, and 400 μl of methanol (HPLC grade, J.T baker) was added to the remaining mixture, followed by centrifugation at 10° C. and at a speed of 13,000 rpm for 20 minutes. Because a protein pellet was formed between a chloroform layer and a water/methanol layer, a supernatant was discarded, and the remaining protein pellet was dried in a hood. The dried protein pellet was dissolved in 20 μl of a solution (94.8% distilled water, 5% (v/v) acetonitrile (ACN) (J.T. Baker), and 0.2% (v/v) formic acid (WAKO)) prior to mass spectrometric analysis.

3. Top-Down Identification of Differentially Expressed Proteins by Mass Spectrometry

The protein samples prepared in 2.3. were subjected to mass spectrometric analysis without digestion using proteases.

For mass spectrometry of proteins, an Orbitrap XL™ (Thermo Fisher) mass spectrometer connected with a nanoLC 2D (Eksigent) system was used. 10 μl of the prepared sample was loaded onto a 3 cm PLRP-S trap column (5 μm, 1000 Å pore size) with an inner diameter of 150 μm, and proteins were separated using a 10 cm PLRP-S Analytical column (5 μm, 1000 Å pore size) with an inner diameter of 75 μm. Conditions for LC used are as follows:

—Mobile Phase:

A: 100% HPLC water, 0.2% (v/v) formic acid (WAKO)

B: 100% (v/v) acetonitrile (J.T. Baker), 0.2% (v/v) formic acid (WAKO)

—Flow Rate: 350 nL/Min

—Gradient: B 5%→20% (5 min), 20%→21% (5 min), 21%→30% (45 min), 30%→40% (15 min), 40%→52% (8 min), 52%→85% (5 min), 85%→5% (5 min), 5%→5% (12 min).

Mass spectrometry data were processed and identified by a ProSightPC program (version 3.0). The relative abundances of proteins were calculated using peak areas from the Extracted Ion Chromatograms (XICs). The peak areas from XICs were manually integrated using peak integration software in Xcalibur for the most abundant isotopic peaks of the proteins. Differential expression of CXCL7 protein was observed in the plasma of colorectal cancer patients, compared to the plasma of normal persons. A CXCL7 expression ratio of a colorectal cancer patient to a normal person was calculated and given in Table 1.

TABLE 1

| | | | Expression ratio |
|---|---|---|---|
| | | | (colorectal cancer |
| | | Molecular | group/normal |
| Protein | Amino acid sequence | weight (Da) | group) |
| CXCL7 | N- | 10255.38 | About 3.0 |
| amino acids | SSTKGQTKRNLAKGKEESLDSDLYAELRC*M | | |
| 35-128 | C*IKTTSGIHPKNIQSLEVIGKGTHC*NQVEVIA | | |
| | TLKDGRKIC*LDPDAPRIKKIVQKKLAGDESAD- | | |
| | C (SEQ ID NO: 1) | | |
| CXCL7 | N- | | |
| amino acids | NLAKGKEESLDSDLYAELRC*MC*IKTTSGIHP | 9297.81 | About 5.6 |
| 44-128 | KNIQSLEVIGKGTHC*NQVEVIATLKDGRKIC*L | | |
| | DPDAPRIKKIVQKKLAGDESAD-C (SEQ ID NO: | | |
| | 2) | | |

“*”refers to a cysteine residue linked by a disulfide bond.

A precursor of the CXCL7 protein is a protein composed of 128 amino acids, and a protein at amino acid positions 1 to 128 (Uniprot No. P02775), where a signal peptide is a polypeptide at amino acid positions 1 to 34, and the CXCL7 protein is a protein at amino acid positions 35 to 128. As shown in Table 1, the amount of the CXCL7 protein of SEQ ID NO: 1 was increased about 3.0 times in the colorectal cancer group, compared to the normal group.

A CXCL7 protein having an amino acid sequence of SEQ ID NO: 2 is a protein at amino acid positions 44 to 128. As shown in Table 1, the amount of the CXCL7 protein of SEQ ID NO: 2 was increased about 5.6 times in the colorectal cancer group, compared to the normal group.

Consequently, CXCL7 protein was confirmed as a diagnostic marker for colorectal cancer.

4. Verification of Differential Expression of CXCL7 Protein

To verify expression patterns of the CXCL7 protein, plasma samples were obtained from 32 normal persons and 75 colorectal cancer patients (Stage 1: 14 patients, Stage 2: 14 patients, Stage 3: 33 patients, and Stage 4: 14 patients).

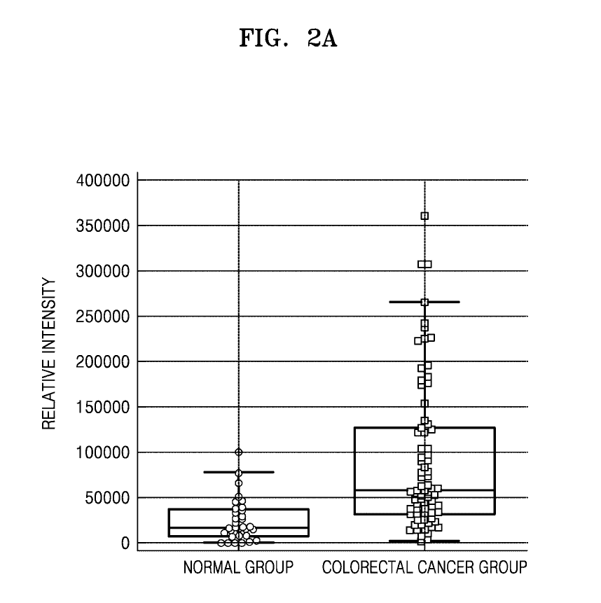

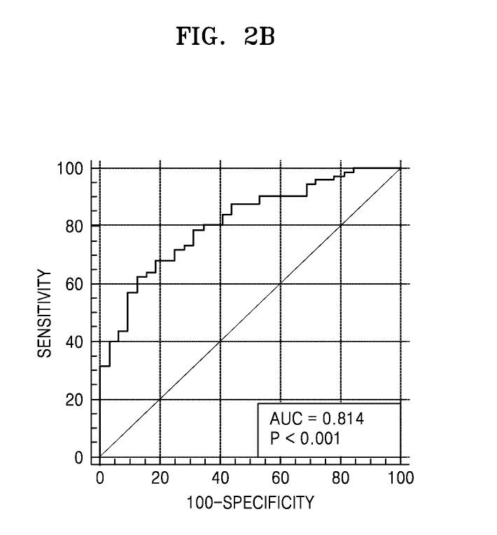

Each 50 μg of the plasma samples was electrophoresed on a 15% (w/v) SDS-polyacrylamide gel. The electrophoresed gel was transferred onto a PVDF membrane, and immunoblotting was performed using an anti-CXCL7 antibody (Santacruz biotechnology) followed by a secondary antibody (Santacruz biotechnology). From the results of immunoblotting, relative intensities of the CXCL7 protein were calculated, and the results are shown in FIG. 2A. Further, calculated CXCL7 protein concentrations were presented as a receiver operating characteristic (ROC) curve of FIG. 2B.

As shown in FIG. 2A, higher intensities of CXCL7 protein were observed in the colorectal cancer group, compared to the normal group. Further, as shown in FIG. 2B, the area under curve (AUC) of CXCL7 protein was 0.814 (p<0.001), indicating that CXCL7 protein is a marker with high diagnostic sensitivity and specificity for colorectal cancer.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. A method of diagnosing colorectal cancer, the method comprising:
measuring an expression level of a chemokine (C-X-C motif) ligand 7 (CXCL7) protein or a fragment thereof in a biological sample separated from a subject suspected of having colorectal cancer; and
comparing the measured expression level with a protein expression level of a normal control group.

2. The method of claim 1, wherein the CXCL7 protein comprises amino acid sequences at positions 35 to 128, or amino acid sequences at positions 44 to 128 from the N-terminus thereof.

3. The method of claim 1, wherein the CXCL7 protein comprises an amino acid sequence of SEQ ID NO: 1 or 2.

4. The method of claim 1, wherein the CXCL7 protein or a fragment thereof is a CXCL7 isoform protein.

5. The method of claim 1, wherein the biological sample is blood, plasma, serum, bone marrow fluid, lymphatic fluid, saliva, lachrymal fluid, mucosal fluid, amniotic fluid, or a combination thereof.

6. The method of claim 1, wherein the biological sample comprises an intact protein.

7. The method of claim 1, wherein the measuring comprises incubating the biological sample and an antibody specifically binding to the CXCL7 protein or the fragment thereof.

8. The method of claim 7, wherein the antibody is a polyclonal antibody or a monoclonal antibody.

9. The method of claim 1, wherein the measuring comprises performing mass spectrometry of the biological sample.

10. The method of claim 9, wherein the performing mass spectrometry of the biological sample is performing mass spectrometry of an intact protein.


SEQUENCE LISTING (2 sequences)

SEQ ID NO: 1 - protein, 94 aa, Artificial Sequence
  Human CXCL7 protein(amino acid position 35-128)
SSTKGQTKRNLAKGKEESLDSDLYAELRCMCIKTTSGIHPKNIQSLEVIGKGTHCNQVEVIATLKDGRKICLDPDAPRIKKIVQKKLAGDESAD

SEQ ID NO: 2 - protein, 85 aa, Artificial Sequence
  Human CXCL7 protein(amino acid position 44-128)
NLAKGKEESLDSDLYAELRCMCIKTTSGIHPKNIQSLEVIGKGTHCNQVEVIATLKDGRKICLDPDAPRIKKIVQKKLAGDESAD